(19) United States
(12) Patent Application Publication
(10) Pub. No.: US 2026/0020874 A1
(43) Pub. Date: Jan. 22, 2026

(54) TREATMENT TOOL AND PAD

(71) Applicant: OLYMPUS MEDICAL SYSTEMS CORP., Tokyo (JP)

(72) Inventors: Keisuke Nagao, Tokyo (JP); Marina Asahina, Sagamihara-shi (JP); Kaito Sato, Tokyo (JP); Tsubasa Niiyama, Tokyo (JP)

(21) Appl. No.: 18/990,492

(22) Filed: Dec. 20, 2024

Related U.S. Application Data
(63) Continuation of application No. PCT/JP2024/025706, filed on Jul. 17, 2024.

(51) Int. Cl. A61B 17/32; A61B 18/14; A61B 18/00

(52) U.S. Cl. CPC A61B 17/320092; A61B 18/1445; A61B 2018/00083; A61B 2018/00601; A61B 2018/0063

(57) ABSTRACT

A treatment tool includes: an ultrasonic blade that includes a treatment face having a first face and having a second face adjacent to the first face; a gripper that includes an electrode configured to open and close with respect to the ultrasonic blade to grip a body tissue between the gripper and the ultrasonic blade; an abutting portion that is made of an electrically insulating material and is disposed in the gripper, the abutting portion being configured to abut against the first face when the gripper closes with respect to the ultrasonic blade; and a protruding portion that is made of an electrically insulating material, is spaced apart from the abutting portion in the gripper, and protrudes toward the second face.

### CROSS REFERENCES TO RELATED APPLICATIONS

This application is a continuation of International Application No. PCT/JP2024/025706, filed on July 17, 2024, the entire contents of which are incorporated herein by reference.

### BACKGROUND

### Technical Field

The present disclosure relates to a treatment tool and a pad.

### Related Art

In the related art, a treatment tool is known that supplies ultrasonic vibrations and a high-frequency current as the treatment energy to the part to be treated in the body tissue (hereinafter, referred to as the treatment target), and accordingly treats the treatment target (for example, refer to Japanese Patent Application Laid-open No. 2016-73729).

The treatment tool disclosed in Patent Literature 1 includes an ultrasonic blade and a gripper. The ultrasonic blade supplies ultrasonic vibrations and a high-frequency current to the treatment target. The gripper opens and closes with respect to the ultrasonic blade, so that the treatment target can be gripped in between the gripper and the ultrasonic blade. Moreover, the gripper supplies a high-frequency current to the treatment target.

### SUMMARY

In some embodiments, a treatment tool includes: an ultrasonic blade that includes a treatment face having a first face and having a second face adjacent to the first face, the ultrasonic blade being configured to supply ultrasonic vibration and high-frequency current from the treatment face to a body tissue; a gripper that includes an electrode configured to open and close with respect to the ultrasonic blade to grip the body tissue between the gripper and the ultrasonic blade and supply the high-frequency current to the gripped body tissue; an abutting portion that is made of an electrically insulating material and is disposed in the gripper, the abutting portion being configured to abut against the first face when the gripper closes with respect to the ultrasonic blade; and a protruding portion that is made of an electrically insulating material, is spaced apart from the abutting portion in the gripper, and protrudes toward the second face, a part of the electrode being exposed from a region between the abutting portion and the protruding portion and facing the ultrasonic blade, and the abutting portion and the protruding portion forming a pad having an integrated structure.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram for explaining a configuration of the front end portion in a treatment tool;

FIGS. 3A to 3C are diagrams for explaining a configuration of a gripper;

FIGS. 4A to 4C are diagrams for explaining a configuration of a jaw;

FIGS. 5A to 5D are diagrams illustrating a configuration of a pad;

FIGS. 6A and 6B are diagrams for explaining a structure by which the pad is attached to the jaw;

FIGS. 7A to 7C and 8A to8D are diagrams for explaining a first modification example of the embodiment;

FIGS. 9, 10A to 10C, 11A, and 11B are diagrams for explaining a second modification example of the embodiment; and

FIGS. 12A to 12C, 13A to 13C, 14A to 14C, and 15A to 15C are diagrams for explaining a fourth modification example of the embodiment.

### DETAILED DESCRIPTION

An illustrative embodiment of the present invention (hereinafter, called an "embodiment") is described below with reference to the accompanying drawings. However, the present invention is not limited by an embodiment described below. Moreover, in the drawings, identical constituent elements are referred to by the same reference numerals.

Overall configuration of treatment system

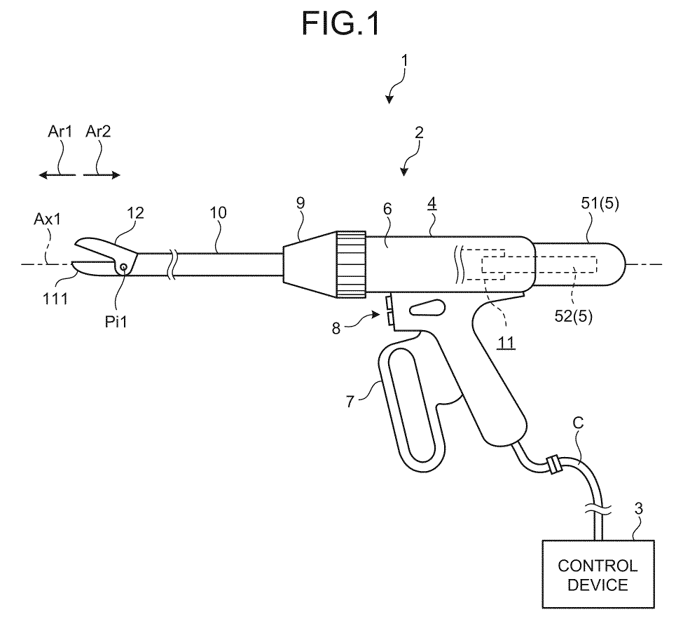

FIG. 1 is a diagram illustrating a treatment system 1 according to the embodiment. The treatment system applies treatment energy to the part to be treated in the body tissue (hereinafter, referred to as the treatment target) and accordingly treats the treatment target. The treatment energy according to the present embodiment represents ultrasonic energy and high-frequency energy. Meanwhile, the treatment that can be carried out by the treatment system 1 according to the present embodiment includes solidification (sealing) of the treatment target or dissection of the treatment target. Alternatively, solidification and dissection can be performed in a simultaneous manner. As illustrated in FIG. 1, the treatment system 1 includes a treatment tool 2 and a control device 3.

### Configuration of treatment tool

In the following explanation, one side along a central axis Ax1 of an outer pipe 10 (see FIG. 1) is referred to as a front end side Ar1 and the other side is referred to as a proximal end side Ar2. Moreover, the term "width direction" mentioned below represents a direction orthogonal to both of the central axis Ax1 and a direction in which a gripper 12 opens and closes with respect to a treatment portion 111, and implies a direction orthogonal to a plane in which FIG. 1 is illustrated.

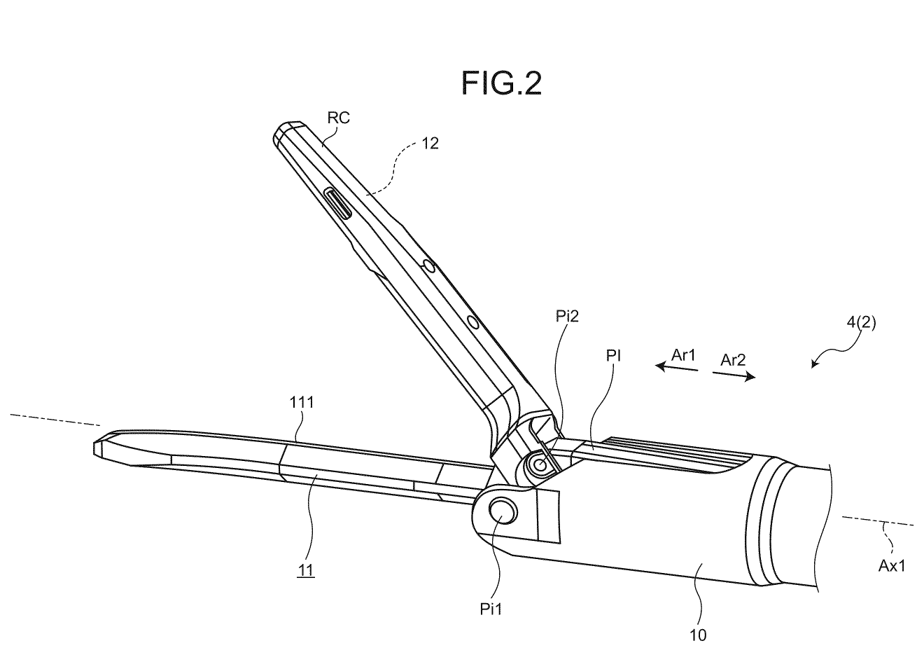

FIG. 2 is a diagram for explaining a configuration of the front end portion in the treatment tool 2. More particularly, FIG. 2 is a perspective view illustrating the front end portion of the treatment tool 2. The treatment tool 2 applies ultrasonic energy and high-frequency energy to the treatment target, and accordingly treats the treatment target. As illustrated in FIG. 1, the treatment tool 2 includes a handpiece 4 and an ultrasonic transducer 5.

As illustrated in FIGS. 1 and 2, the handpiece 4 includes a fixed handle 6 (see FIG. 1), an operation handle 7 (see FIG. 1), a switch 8 (see FIG. 1), a rotation knob 9 (see FIG. 1), the outer pipe 10, an ultrasonic blade 11, and the gripper 12.

The fixed handle 6 supports the entire treatment tool 2 and represents the portion that is gripped by the operator (user) such as a technician.

The operation handle 7 is attached to the fixed handle 6 in a movable manner, and receives opening-closing operations performed by the operator such as a technician.

The switch 8 is disposed in an exposed state on the outside of the fixed handle 6, and receives treatment operations performed by the operator such as a technician.

The rotation knob 9 has a substantially cylindrical shape that is coaxial to the central axis Ax1, and is disposed on the front end side Ar1 of the fixed handle 6. The rotation knob 9 receives rotation operations performed by the operator such as a technician. In response to a rotation operation, the rotation knob 9 rotates with respect to the fixed handle 6 around the rotation axis Ax1. Moreover, due to the rotation of the rotation knob 9; the outer pipe 10, the ultrasonic blade 11, and the gripper 12 rotate around the central axis Ax1.

The outer pipe 10 is a cylindrical pipe made of an electrically conductive material such as a metal. The end portion of the outer pipe 10 on the proximal end side Ar2 is fixed to the rotation knob 9.

In the outer pipe 10, at the end portion on the front end side Ar1, a first pin Pi1 (see FIGS. 1 and 2) that has a columnar shape extending in the direction orthogonal to the plane in which FIG. 1 is illustrated, that engages with the gripper 12, and that supports the gripper 12 in a pivotable manner is fixed. In the present embodiment, the first pin Pi1 is made of an electrically conductive material such as a metal.

The outer periphery of the outer pipe 10 is covered by an outer tube (not illustrated) that is made of an electrically insulating material. Inside the outer pipe 10, a tubelike inner pipe PI (see FIG. 2) is inserted that reciprocates along the longitudinal direction of the outer pipe 10 according to an opening-closing action of the operation handle 7 performed by the operator such as a technician. Moreover, at the end portion of the inner pipe P1 on the front end side Ar1, a second pin Pi2 (see FIG. 2) is fixed that has a columnar shape extending in the direction orthogonal to the plane in which FIG. 1 is illustrated and that engages with the gripper 12. In the present embodiment, in FIG. 2, with reference to the first pin Pi1, the second pin Pi2 is disposed on the upper side (i.e., the side on which the gripper 12 is disposed with respect to the treatment portion 111).

The ultrasonic blade 11 is made of an electrically conductive material and has an elongated shape extending along the central axis Ax1. Moreover, in which the treatment portion 111 is protruding to the outside, the ultrasonic blade 11 is inserted inside the inner pipe P1 in the state. At that time, as illustrated in FIG. 1, the end portion of the ultrasonic blade 11 on the proximal end side Ar2 gets mechanically connected to an ultrasonic vibrator 52 that constitutes the ultrasonic transducer 5. Then, the ultrasonic blade 11 transmits ultrasonic vibrations, which are generated by the ultrasonic transducer 5, from the end portion thereof on the proximal end side Ar2 to the treatment portion 111. The ultrasonic vibrations are longitudinal vibrations occurring along the central axis Ax1. Meanwhile, in the ultrasonic blade 11, the outer periphery excluding the treatment portion 111 is covered by an inner tube (not illustrated) that is made of an electrically insulating material.

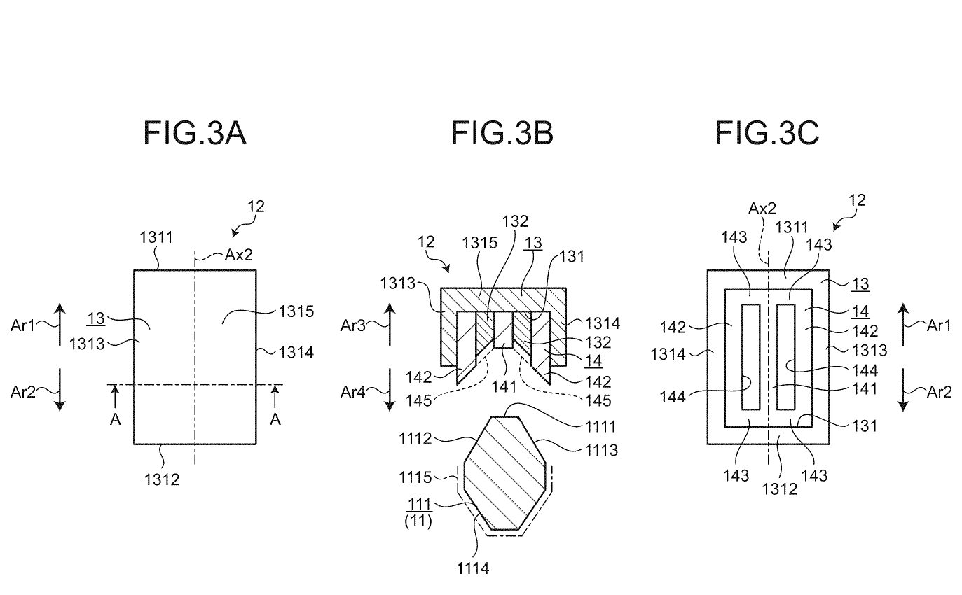

In the present embodiment, the cross-sectional surface when the treatment portion 111 is cut along the plane orthogonal to the central axis Ax1 has a substantially octagonal shape (refer to FIG. 3B). The octagonal shape of the cross-sectional surface of the treatment portion 111 is only exemplary, and it is possible to have some other cross-sectional shape. In the following explanation, for explanatory convenience, the treatment portion 111 is assumed to have an octagonal cross-sectional shape.

In the following explanation, in the treatment portion 111, the flat face positioned on the side of the gripper 12 is referred to as an abutting face 1111. When the gripper 12 closes with respect to the treatment portion 111, the abutting face 1111 abuts against an abutting portion 141 of a pad 14 that constitutes the gripper 12. The abutting face 1111 represents a first face. Moreover, in the treatment portion 111, the faces that are adjacent to the abutting face 1111 in the circumferential direction around the central axis and that, as the distance from the abutting face 1111 increases, become inclined toward the sides present away from the gripper 12 are referred to as inclined faces 1112 and 1113. The inclined faces 1112 and 1113 represent second faces. Moreover, the abutting face 1111 and the inclined faces 1112 and 1113 represent treatment faces. Furthermore, in the treatment portion 111, the face other than the abutting face 1111 and the inclined faces 1112 and 1113 is referred to as a non-treatment face 1114.

The non-treatment face 1114 is covered by a coating layer 1115 that is made of an electrically insulating material. As the material constituting the coating layer 1115, polyetheretherketone (PEEK) can be cited as an example.

When the treatment target is treated using ultrasonic vibrations, there occurs an increase in the temperature of the non-treatment face 1114, which is the face other than the abutting face 1111 and the inclined faces 1112 and 1113 representing the treatment faces in the treatment portion 111. In the state in which the temperature of the non-treatment face 1114 has increased, if the non-treatment face 1114 comes in contact with a part other than the treatment target in the body tissue, it results in an unintended impact on the body tissue. That is, in the present embodiment, since the non-treatment face 1114 is covered by the coating layer 1115, it becomes possible to avoid an unintended impact on the body tissue.

The gripper 12 is connected to the outer pipe 10 using the first pin Pi1. Moreover, the gripper 12 is connected to the inner pipe PI using the second pin Pi2. Thus, in tandem with the reciprocation of the inner pipe PI according to an opening-closing action of the operation handle 7 performed by the operator such as a technician, the gripper 12 pivots with respect to the outer pipe 10 around the first pin Pi1. It results in the opening and closing of the gripper 12 with respect to the treatment portion 111, and the treatment target can be gripped in between the gripper 12 and the treatment portion 111.

Meanwhile, the treatment tool 2 can be configured as a close-by-pressing type tool or as a close-by-pulling type tool.

The treatment tool 2 of the close-by-pressing type has the following configuration. In tandem with the movement of the inner pipe PI toward the front end side Ar1, the gripper 12 pivots around the first pin Pi1 and in the direction approaching the treatment portion 111. That is, the gripper 12 closes with respect to the treatment portion 111. Moreover, in tandem with the movement of the inner pipe PI toward the proximal end side Ar2, the gripper 12 pivots around the first pin Pi1 and in the direction moving away from the treatment portion 111. That is, the gripper 12 opens with respect to the treatment portion 111.

The treatment tool 2 of the close-by-pulling type has the following configuration. In tandem with the movement of the inner pipe PI toward the proximal end side Ar2, the gripper 12 pivots around the first pin Pi1 and in the direction approaching the treatment portion 111. That is, the gripper 12 closes with respect to the treatment portion 111. Moreover, in tandem with the movement of the inner pipe PI toward the front end side Ar1, the gripper 12 pivots around the first pin Pi1 and in the direction moving away from the treatment portion 111. That is, the gripper 12 opens with respect to the treatment portion 111.

Regarding the detailed configuration of the gripper 12, the explanation is given later in a section “Regarding configuration of gripper”.

As illustrated in FIG. 1, the ultrasonic transducer 5 includes a TD (transducer) case 51 and an ultrasonic vibrator 52.

The TD case 51 supports the ultrasonic vibrator 52 and is connected to the fixed handle 6 in a detachably attachable manner.

Under the control performed by the control device 3, the ultrasonic vibrator 52 generates ultrasonic vibrations. In the present embodiment, the ultrasonic vibrator 52 is configured using a bolt-clamped Langevin type transducer (BLT).

### Configuration of control device

The control device 3 comprehensively controls the operations of the treatment tool 2 via an electric cable C (see FIG. 1). More particularly, via the electric cable C, the control device 3 detects a treatment operation that is performed by the operator, such as a technician, using the switch 8. When a treatment operation is detected, the control device 3 applies the treatment energy to the treatment target, which is being gripped in between the gripper 12 and the treatment portion 111, via the electric cable C. That is, the control device 3 treats the treatment target.

For example, at the time of applying the ultrasonic energy to the treatment target, the control device 3 supplies a driving power to the ultrasonic vibrator 52 via the electric cable C. As a result, the ultrasonic vibrator 52 generates longitudinal vibrations occurring along the central axis Ax1 (i.e., generates ultrasonic vibrations). According to those longitudinal vibrations, the treatment portion 111 vibrates at the desired amplitude. Then, the ultrasonic vibrations are supplied from the treatment portion 111 to the treatment target, which is being gripped between the gripper 12 and the treatment portion 111. In other words, the ultrasonic energy is applied to the gripper 12.

Moreover, for example, at the time of applying the high-frequency energy to the treatment target, the control device 3 follows, for example, the conductive path including the outer pipe 10, the first pin Pi1, and the gripper 12 via the electric cable C, and supplies the high-frequency power in between the gripper 12 and the ultrasonic blade 11. When the high-frequency power is supplied in between the gripper 12 and the ultrasonic blade 11, a high-frequency current is supplied to the treatment target that is being gripped between the gripper 12 and the treatment portion 111. In other words, the high-frequency energy is applied to the treatment target.

### Regarding configuration of gripper

Given below is the explanation about a configuration of the gripper 12. In the following explanation about a configuration of the gripper 12, the side away from the treatment portion 111 is referred to as a backside Ar3 (see FIG. 3B), and the side close to the treatment portion 111 is referred to as a treatment portion side Ar4 (see FIG. 3B). FIGS. 3A to 3C are diagrams for explaining a configuration of the gripper 12. More particularly, FIG. 3A is a diagram illustrating the gripper 12 when viewed from the backside Ar3. Moreover, FIG. 3B is a cross-sectional view of an A-A line illustrated in FIG. 3A. Furthermore, FIG. 3C is a diagram illustrating the gripper 12 when viewed from the treatment portion side Ar4. As illustrated in FIGS. 3A to 3C, the gripper 12 includes a jaw 13 and the pad 14.

Regarding configuration of jaw

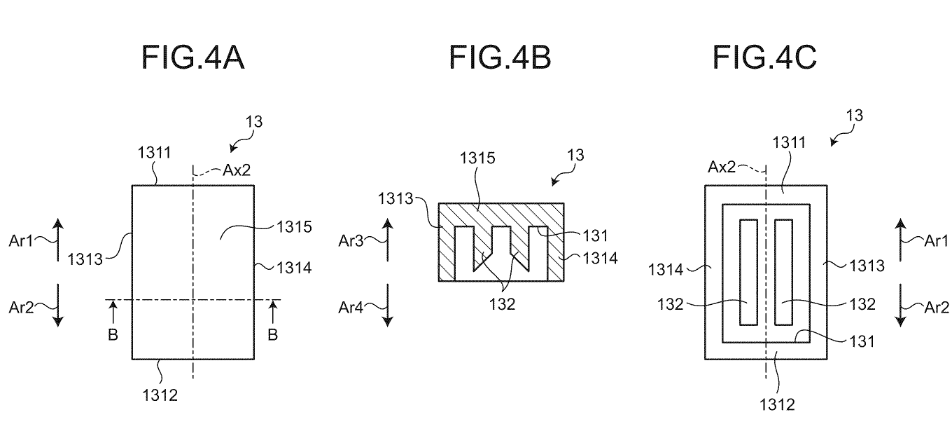

FIGS. 4A to 4C are diagrams for explaining a configuration of the jaw 13. More particularly, FIG. 4A is a diagram illustrating the jaw 13 when viewed from the backside Ar3. Moreover, FIG. 4B is a cross-sectional view of a B-B line illustrated in FIG. 4A. Furthermore, FIG. 4C is a diagram illustrating the jaw 13 when viewed from the treatment portion side Ar4. The jaw 13 is connected to the outer pipe 10 using the first pin Pi1, is connected to the inner pipe PI using the second pin Pi2, and performs the opening-closing action with respect to the treatment portion 111. The jaw 13 is made of an electrically conductive material such as a metal, and functions as an electrode that supplies a high-frequency current to the treatment target. Moreover, in the jaw 13, on the face of the backside Ar3, a cover RC (see FIG. 2) made of an electrically insulating material is formed in an integrated manner for covering the face of the backside Ar3. For that reason, in the jaw 13 functioning as the electrode, the face on the backside Ar3 not functioning as the treatment face is covered by the cover RC, and it becomes possible to avoid any unintended impact on the body tissue from the backside Ar3. Meanwhile, in the present embodiment, although the cover RC is insert-molded into the jaw 13, that is not the only possible case. Alternatively, for example, the cover RC can be snap-fit to the jaw 13 or can be fixed to the jaw 13 using a metallic pin.

The jaw 13 has an elongated shape extending in the direction along the central axis Ax1. In the following explanation, the axis that passes through the center of the jaw 13 in the width direction and that is parallel to the central axis Ax1 is referred to as a central axis Ax2 (see FIGS. 3A to 5D).

As illustrated in FIGS. 4B and 4C, in the jaw 13, on the face present on the treatment portion side Ar4, a concave portion 131 is provided that is depressed toward the backside Ar3 and that extends along the longitudinal direction of the jaw 13. The concave portion 131 is surrounded by sidewall portions 1311 to 1314, which are positioned on the front end side Ar1, the proximal end side Ar2, and both sides in the width direction; and by a bottom wall portion 1315 of the backside Ar3. Moreover, as illustrated in FIG. 4C, when viewed from the treatment portion side Ar4, the concave portion 131 has a shape that is symmetrical with reference to the central axis Ax2.

On the face present on the treatment portion side Ar4 of the bottom wall portion 1315, a pair of jaw-side protruding portions 132 are erected as illustrated in FIGS. 4B and 4C. As illustrated in FIG. 4C, when viewed from the treatment portion side Ar4, the pair of jaw-side protruding portions 132 are disposed to be symmetrical with reference to the central axis Ax2. More particularly, the jaw-side protruding portions 132 extend in a linear manner along the central axis Ax2. As illustrated in FIG. 4B, the height of the jaw-side protruding portions 132 is shorter than the height of the sidewall portions 1311 to 1314. Moreover, as illustrated in FIG. 4B, the tip of each jaw-side protruding portion 132 is made of an inclined face that is inclined toward the treatment portion side Ar4 as the distance from the other jaw-side protruding portion 132 goes on increasing.

### Regarding configuration of pad

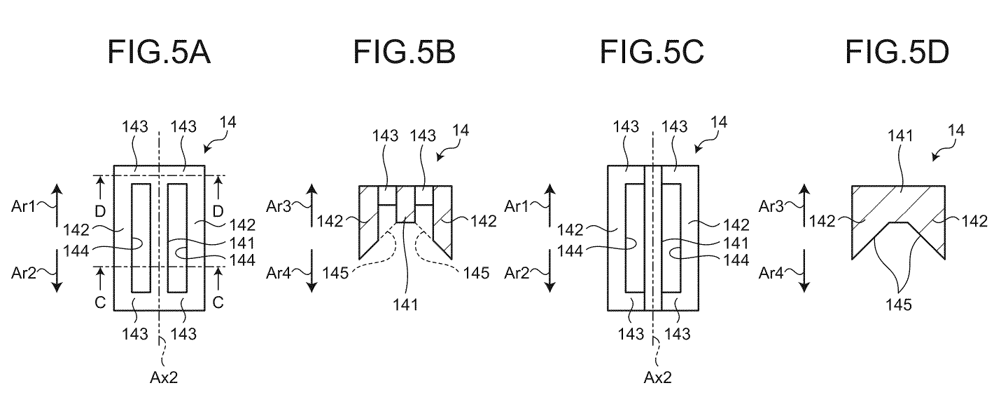

FIGS. 5A to 5D are diagrams illustrating a configuration of the pad 14. More particularly, FIG. 5A is a diagram illustrating the pad 14 when viewed from the backside Ar3. Moreover, FIG. 5B is a cross-sectional view of a C-C line illustrated in FIG. 5A. Furthermore, FIG. 5C is a diagram illustrating the pad 14 when viewed from the treatment portion side Ar4. Moreover, FIG. 5D is a cross-sectional view of a D-D line illustrated in FIG. 5A. The pad 14 is made of a material having electrical insulation properties and biocompatibility, such as polytetrafluoroethylene (PTFE). In the state of being engaged inside the concave portion 131, the pad 14 is attached to the jaw 13. Regarding the structure by which the pad 14 is attached to the jaw 13, the explanation is given later in a section “Regarding attachment structure of pad with respect to jaw”.

As illustrated in FIGS. 5A to 5D, the pad 14 includes an abutting portion 141, a pair of pad-side protruding portions 142, and four connecting portions 143.

As illustrated in FIGS. 5A and 5C, when viewed from the backside Ar3 or the treatment portion side Ar4, the abutting portion 141 is positioned on the central axis Ax2 and extends in a linear manner along the central axis Ax2. Moreover, when viewed from the backside Ar3 or the treatment portion side Ar4, the abutting portion 141 has a shape that is symmetrical with reference to the central axis Ax2. As illustrated in FIG. 3C, the length of the abutting portion 141 in the longitudinal direction is slightly shorter than the distance between the sidewall portions 1311 and 1312. Moreover, as illustrated in FIG. 5B, the end face of the abutting portion 141 on the treatment portion side Ar4 is a flat face running along the width direction. Moreover, as illustrated in FIG. 3B, when the pad 14 is attached to the jaw 13, the end face of the abutting portion 141 on the treatment portion side Ar4 is positioned in between the inclined faces representing the tips of the pair of jaw-side protruding portions 132. When the gripper 12 closes with respect to the treatment portion 111, the abutting portion 141 abuts against the abutting face 1111.

As illustrated in FIGS. 5A and 5C, when viewed from the backside Ar3 or the treatment portion side Ar4, the pair of pad-side protruding portions 142 are positioned on both sides of the abutting portion 141 in the width direction and extend in a linear manner along the central axis Ax2. The pair of pad-side protruding portions 142 are disposed to be symmetrical with reference to the central axis Ax2. The length of the pad-side protruding portions 142 in the longitudinal direction is same as the length of the abutting portion 141 in the longitudinal direction. Moreover, as illustrated in FIG. 5B, the end face of each pad-side protruding portion 142 on the treatment portion side Ar4 is made of an inclined face that becomes inclined toward the treatment portion side Ar4 as the distance from the other pad-side protruding portion 142 goes on increasing. Moreover, as illustrated in FIG. 3B, in the state in which the pad 14 is attached to the jaw 13, the end faces of the pad-side protruding portions 142 on the treatment portion side Ar4 are positioned on both sides of the pair of jaw-side protruding portions 132 in the width direction, and are positioned more on the treatment portion side Ar4 with respect to the end faces of the sidewall portions 1311 to 1314 on the treatment portion side Ar4. The pair of pad-side protruding portions 142 face the inclined faces 1112 and 1113, respectively. That is, the pair of pad-side protruding portions 142 are spaced apart from the abutting portion 141, and protrude toward the inclined faces 1112 and 1113, respectively. The pair of pad-side protruding portions 142 represent protruding portions.

The four connecting portions 143 connect the front end side Ar1 and the proximal end side Ar2 of the end portion of the abutting portion 141 on the backside Ar3 with the front end side Ar1 and the proximal end side Ar2 of the end portion of the pair of pad-side protruding portions 142 on the backside Ar3. As a result, as illustrated in FIGS. 5A and 5C, in the pad 14, two opening portions 144 that are surrounded by the abutting portion 141, the pair of pad-side protruding portions 142, and four connecting portions 143 are formed. In the two opening portions 144, the pair of jaw-side protruding portions 132 are inserted.

As illustrated in FIG. 5D, on the face of the pad 14 on the treatment portion side Ar4, guides 145 are provided at the end portions on the front end side Ar1 and the proximal end side Ar2. For explanatory convenience, in FIGS. 3B and 5B, the guides 145 are illustrated using dashed lines.

The guides 145 are provided at the end portion on the front end side Ar1 and the end portion on the proximal end side Ar2 of the end face of the pair of pad-side protruding portions 142 on the treatment portion side Ar4; are provided on the end faces of the four connecting portions 143 on the treatment portion side Ar4; and are identical inclined faces to the inclined faces representing the end faces of the pair of pad-side protruding portions 142 on the treatment portion side Ar4. When the gripper 12 closes with respect to the treatment portion 111, the treatment portion 111 makes sliding contact with the guides 145, so that a relative position between the gripper 12 and the treatment portion 111 in the width direction get determined.

### Regarding attachment structure of pad with respect to jaw

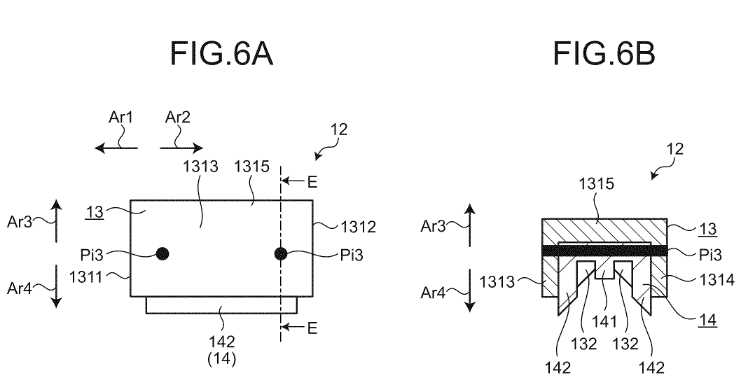

Given below is the explanation of a structure by which the pad 14 is attached to the jaw 13. FIGS. 6A and 6B are diagrams for explaining a structure by which the pad 14 is attached to the jaw 13. More particularly, FIG. 6A is a side view of the gripper 12 when viewed along the width direction. Moreover, FIG. 6B represents a cross-sectional view of an E-E line illustrated in FIG. 6A. In the present embodiment, as illustrated in FIGS. 6A and 6B, the pad 14 is fixed to the jaw 13 using two third pins Pi3. The two third pins Pi3 represent pins. The two third pins Pi3 have a columnar shape and, along the width direction, pass through sidewall portions 1313 and 1314 of the jaw 13 and the end portion of the pad 14 on the backside Ar3. As a result, the jaw 13 and the pad 14 get fixed to each other. Meanwhile, the two third pins Pi3 are press-fit or welded to the gripper 12 (the jaw 13 and the pad 14).

According to the present embodiment described above, the following effects are achieved. In the treatment tool according to the present embodiment, the pad 14 includes: the abutting portion 141 that, when the gripper 12 closes with respect to the treatment portion 111, is positioned at a distance from the abutting portion 141; and the pad-side protruding portion 142 that protrudes toward the inclined faces 1112 and 1113. The pair of jaw-side protruding portions 132 that represent a part of the jaw 13 functioning as the electrode are exposed from the region between the abutting portion 141 and the pad-side protruding portions 142, and face the treatment portion 111. Hence, in between the jaw 13 and the treatment portion 111, the region for supplying a high-frequency current to the treatment target can be limited to the region between the pair of pad-side protruding portions 142 (the central region in the width direction). As a result, it becomes possible to limit the conductive path present between the sidewall portions 1313 and 1314 and the treatment portion 111 in the jaw 13, and to suppress the sticking of the body tissue onto the sidewall portions 1313 and 1314. Moreover, it becomes possible to reduce an unintended impact on the body tissue other than the treatment target.

Particularly, since the abutting portion 141 and the pad-side protruding portions 142 have an integrated structure, it becomes possible to easily attach the abutting portion 141 and the pad-side protruding portions 142 to the jaw 13.

Moreover, in the treatment tool 2 according to the present embodiment, the sidewall portion 1311 on the front end side Ar1, which represents a part of the jaw 13 functioning as the electrode, is exposed from the front end side Ar1 of the abutting portion 141 and the pad-side protruding portions 142, and faces the treatment portion 111. Hence, a high-frequency current can be supplied to the treatment target from the region that not only includes the end faces of the pair of jaw-side protruding portions 132 on the treatment portion side Ar4 but also includes the end face of the sidewall portion 1311 on the treatment portion side Ar4. That is, it becomes possible to increase the region for supplying a high-frequency current to the treatment target, and to sufficiently secure the treatment performance regarding the treatment target.

Furthermore, in the treatment tool 2 according to the present embodiment, the pad 14 includes the guides 145 with which the treatment portion 111 makes sliding contact when the gripper 12 closes with respect to the treatment portion 111, and which determine the relative position between the gripper 12 and the treatment portion 111 in the width direction. Hence, the clearance between the end face of the jaw-side protruding portion 132 on the treatment portion side Ar4 and the inclined faces 1112 and 1113 can be maintained at a constant value, and electrical shorting can be prevented from occurring between the gripper 12 and the ultrasonic blade 11.

### Other embodiments

Although an exemplary embodiment of the present invention was described above, the present invention should not be limited by the embodiment described above. In the embodiment described above, it is also possible to adapt modification examples from a first modification example to a fourth modification example explained below.

### First modification example

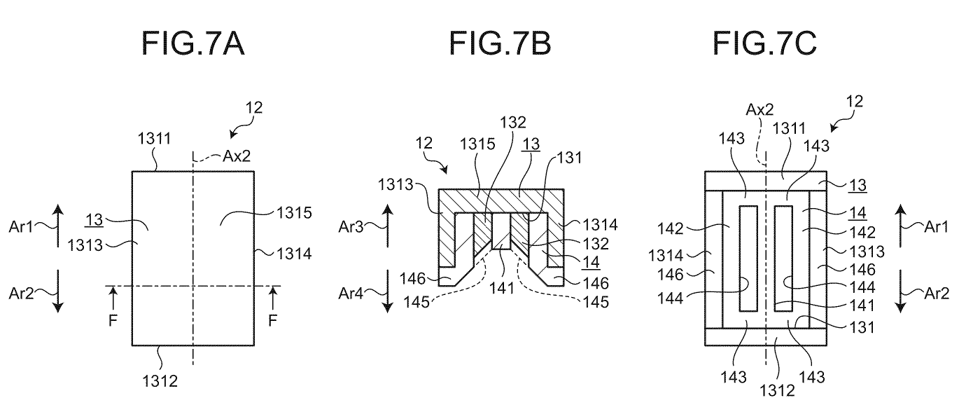

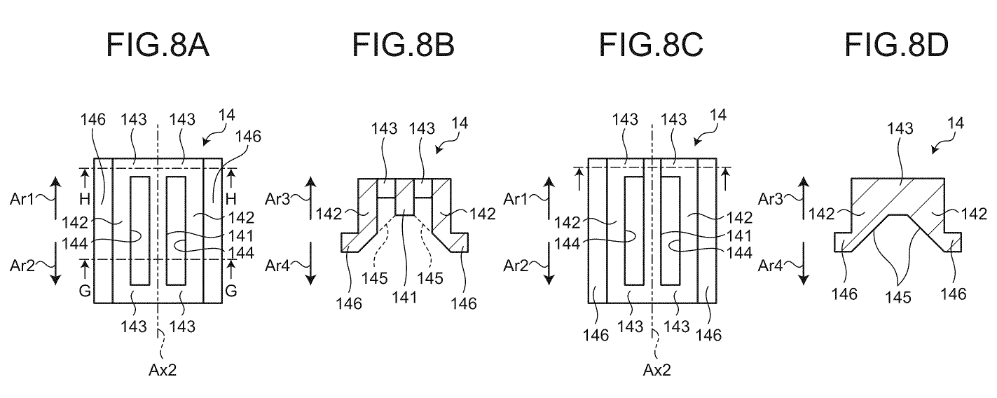

FIGS. 7A to 7C and 8A to8D are diagrams for explaining the first modification example of the embodiment. More particularly, FIGS. 7A to 7C correspond to FIGS. 3A to 3C, respectively, and are diagrams for explaining a configuration of the gripper 12 according to the first modification example. FIG. 7A is a diagram illustrating the gripper 12 according to the first modification example when viewed from the backside Ar3. Moreover, FIG. 7B is a cross-sectional view of an F-F line illustrated in FIG. 7A. Moreover, FIG. 7C is a diagram illustrating the gripper 12 according to the first modification example when viewed from the treatment portion side Ar4. FIGS. 8A to 8D correspond to FIGS. 5A to 5D, respectively, and are diagrams for explaining a configuration of the pad 14 according to the first modification example. FIG. 8A is a diagram illustrating the pad 14 according to the first modification example when viewed from the backside Ar3. Moreover, FIG. 8B is a cross-sectional view of a G-G line illustrated in FIG. 8A. Furthermore, FIG. 8C is a diagram illustrating the pad 14 according to the first modification example when viewed from the treatment portion side Ar4. Moreover, FIG. 8D is a cross-sectional view of an H-H line illustrated in FIG. 8A. In the embodiment described above, the shape of the pad can be changed as illustrated in FIGS. 7A to 8D.

As illustrated in FIGS. 7A to 8D, the pad 14 according to the first modification example differs from the pad 14 according to the embodiment in the way that two covering portions 146 are provided.

As illustrated in FIGS. 7A to 8D, the two covering portions 146 extend outward in the width direction from the end portions of the pair of pad-side protruding portions 142 on the treatment portion side Ar4. As illustrated in FIGS. 7B and 7C, in the state in which the pad 14 is attached to the jaw 13, the two covering portions 146 cover the end faces of the sidewall portions 1313 and 1314 on the treatment portion side Ar4.

According to the first modification example explained above, in addition to achieving identical effects to the effects achieved in the embodiment described above, the following effects are also achieved.

The pad 14 according to the first modification example includes the covering portions 146 that cover the end faces of the sidewall portions 1313 and 1314 on the treatment portion side Ar4.

Hence, the conductive path between the sidewall portions 1313 and 1314 and the treatment portion 111 can be effectively restricted, and the sticking of the body tissue onto the sidewall portions 1313 and 1314 can be effectively suppressed.

### Second modification example

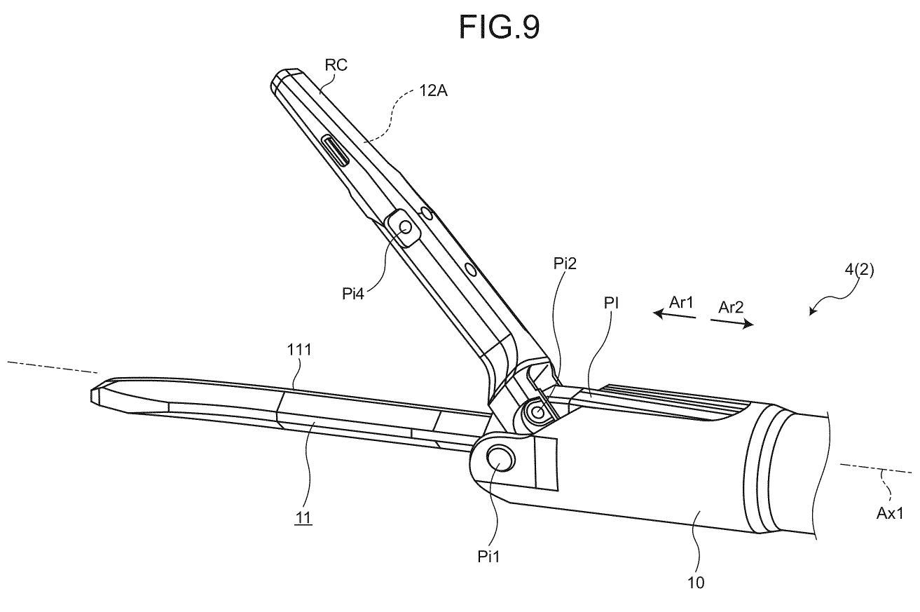

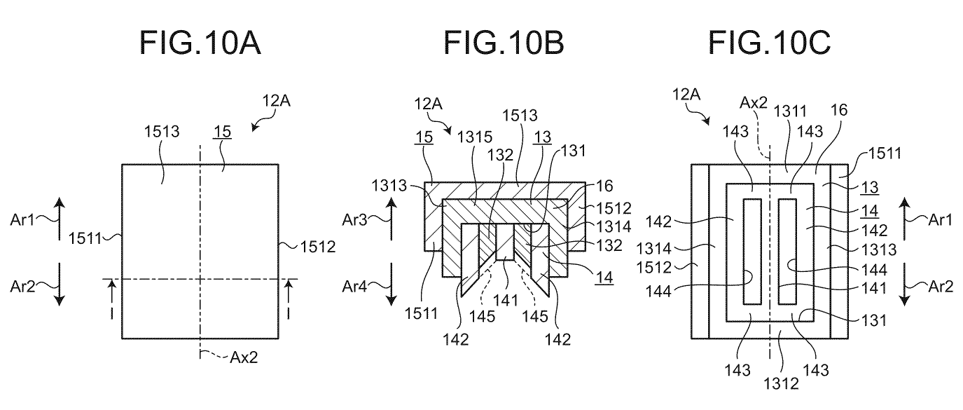

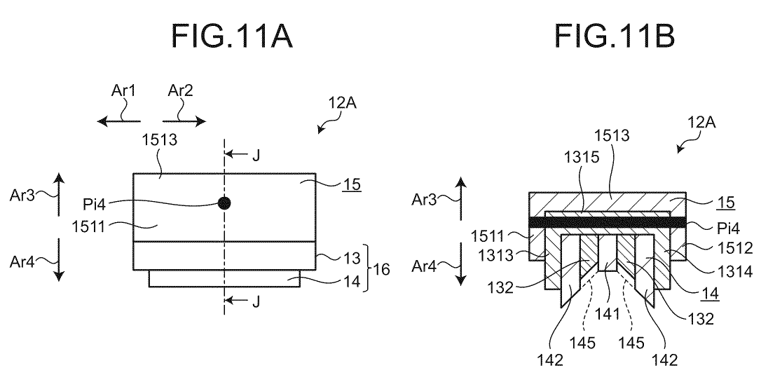

FIGS. 9A to 11B are diagrams forexplaining the second modification example of the embodiment. More particularly, FIG. 9 corresponds to FIG. 2, and is a diagram for explaining a configuration of the front end portion of the treatment tool 2 according to the second modification example. FIGS. 10A to 10C correspond to FIGS. 3A to 3C, respectively, and are diagrams for explaining a configuration of a gripper 12A according to the second modification example. FIG. 10A is a diagram illustrating the gripper 12A when viewed from the backside Ar3. Moreover, FIG. 10B is a cross-sectional view of an I-I line illustrated in FIG. 10A. Furthermore, FIG. 10C is a diagram illustrating the gripper 12A when viewed from the treatment portion side Ar4. FIGS. 11A and 11B are diagrams for explaining a structure by which a swinging portion 16 is attached to a jaw 15. More particularly, FIG. 11A is a side view of the gripper 12A when viewed along the width direction. Moreover, FIG. 11B is a cross-sectional view of a J-J line illustrated in FIG. 11A.

In the embodiment described earlier, the gripper 12 includes only the jaw 13 as the member meant for holding the pad 14. In the gripper 12A according to the second modification example, the jaw 13 according to the embodiment described earlier functions as a holder. Thus, in the following explanation, the jaw 13 is referred to as a holder 13. Moreover, the gripper 12 according to the embodiment described earlier is referred to as the swinging portion 16. The gripper 12A is further configured by adding a jaw 15 to the gripper 12 according to the embodiment described above. The swinging portion 16 that is configured using the holder 13 and the pad 14, which are connected to each other by the third pin Pi3, is swingably attached to the jaw 15.

The jaw 15 is connected to the outer pipe 10 using the first pin Pi1, is connected to the inner pipe PI using the second pin Pi2, and opens and closes with respect to the treatment portion 111. The jaw 15 is made of an electrically conductive material such as a metal. Moreover, in the jaw 15, on the face on the backside Ar3, the cover RC (see FIG. 9) made of an electrically insulating material is formed in an integrated manner to cover the face of the backside Ar3. For that reason, in the jaw 15 functioning as the electrode, the face on the backside Ar3 not functioning as the treatment face is covered by the cover RC, so that it becomes possible to avoid any unintended impact on the body tissue from the backside Ar3. Meanwhile, in the embodiment, although the cover RC is insert-molded into the jaw 15, that is not the only possible case. Alternatively, for example, the cover RC can be snap-fit to the jaw 15 or can be fixed to the jaw 15 using a metallic pin.

The jaw 15 has an elongated shape extending in the direction along the central axis Ax2. As illustrated in FIGS. 10B and 10C, in the jaw 15, on the face on the treatment portion side Ar4, a concave portion 151 is provided that is depressed toward the backside Ar3 and that extends along the longitudinal direction of the jaw 15. The concave portion 151 passes through the end faces of the jaw 15 on the front end side Ar1 and the proximal end side Ar2. That is, the concave portion 151 is surrounded by sidewall portions 1511 and 1512 on both sides in the width direction and by a bottom wall portion 1513 on the backside Ar3. Moreover, as illustrated in FIG. 10C, when viewed from the treatment portion side Ar4, the concave portion 151 has a shape that is symmetrical with reference to the central axis Ax2. The internal size of the concave portion 151 is slightly larger than the external size of the swinging portion 16 (the holder 13).

As illustrated in FIGS. 10A to 11D, the swinging portion 16 is attached to the jaw 15 in the state of being placed inside the concave portion 151.

More particularly, as illustrated in FIGS. 11A to 11D, the swinging portion 16 is swingably attached to the jaw 15 using a fourth pin Pi4. The fourth pin Pi4 is made of an electrically conductive material such as a metal; has a columnar shape; and connects the jaw 15 and the swinging portion 16 to each other by passing through sidewall portions 1511 and 1512 of the jaw 15 and through the end portion of the holder 13 on the backside Ar3 while avoiding the concave portion 131. Then, the swinging portion 16 is swingably attached to the jaw 15 around the central axis of the fourth pin Pi4. As a result of making the swinging portion 16 swingable around the central axis of the fourth pin Pi4, when the treatment target is gripped in between the gripper 12A and the treatment portion 111, the position for applying the strongest force onto the treatment target is not set on the proximal end side Ar2 of the gripper 12A but is set substantially at the center of the gripper 12A in the longitudinal direction. Thus, a substantially even force is applied onto the treatment target that is gripped in between the gripper 12A and the treatment portion 111.

Meanwhile, the conductive path used at the time of supplying the high-frequency power from the control device 3 includes, for example, the outer pipe 10, the first pin Pi1, the jaw 15, the fourth pin Pi4, and the holder 13.

Even when the configuration according to the second modification example is adapted, it becomes possible to achieve identical effects to the effects achieved according to the embodiment described above.

### Third modification example

In the second modification example explained above, the pad 14 is attached to the holder 13 with the third pin Pi3. However, that is not the only possible case. Alternatively, the pad 14 can be joined to the holder 13 by means of insert molding or welding. At that time, Ni-PTFE plating can be performed on the bottom face of the bottom wall portion 1315 of the holder 13 on the treatment portion side Ar4 and, while increasing the temperature of the holder 13, the pad 14 made of NTFE can be attached onto the Ni-PTFE plating by applying pressure. As a result, it becomes possible to enhance the adhesion of the pad 14 with the holder 13.

Even when the configuration according to the third modification example is adapted, it becomes possible to achieve identical effects to the effects achieved according to the second modification example explained above.

### Fourth modification example

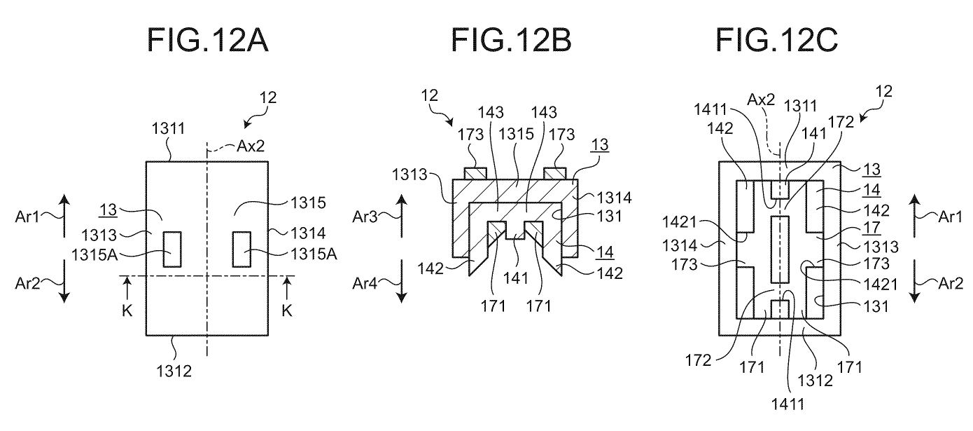

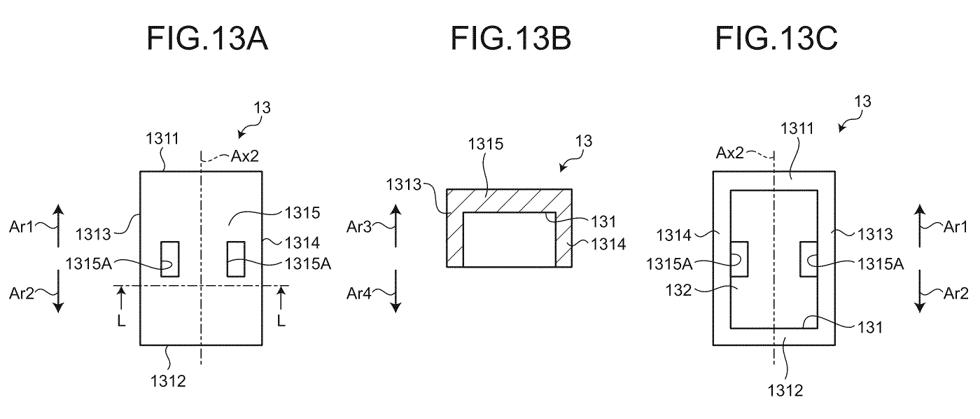

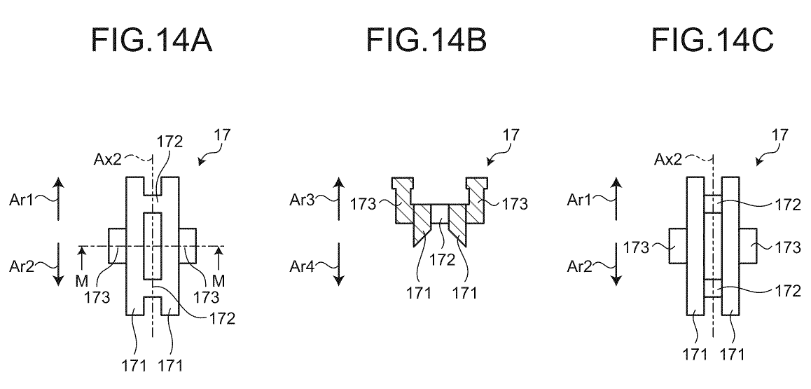

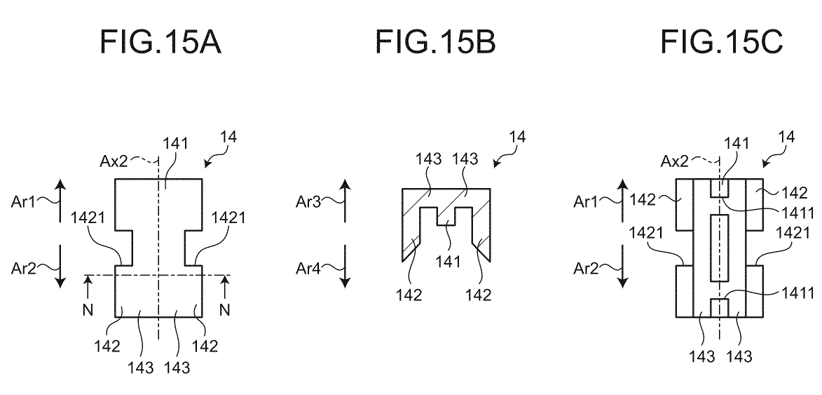

FIGS. 12A to 15C are diagrams for explaining a fourth modification example of the embodiment. More particularly, FIGS. 12A to 12C correspond to FIGS. 3A to 3C, respectively, and is a diagram for explaining a configuration of the gripper 12 according to the fourth modification example. FIG. 12A is a diagram illustrating the gripper 12 according to the fourth modification example when viewed from the backside Ar3. Moreover, FIG. 12B is a cross-sectional view of a K-K line illustrated in FIG. 12A. Furthermore, FIG. 12C is a diagram illustrating the gripper 12 according to the fourth modification example when viewed from the treatment portion side Ar4. FIGS. 13A to 13C are diagrams for explaining a configuration of the jaw 13 according to the fourth modification example. More particularly, FIG. 13A is a diagram illustrating the jaw 13 according to the fourth modification example when viewed from the backside Ar3. Moreover, FIG. 13B is a cross-sectional view of an L-L line illustrated in FIG. 13A. Furthermore, FIG. 13C is a diagram illustrating the jaw 13 according to the fourth modification example when viewed from the treatment portion side Ar4. FIGS. 14A to 14C are diagrams for explaining a configuration of an electrode 17. More particularly, FIG. 14A is a diagram illustrating the electrode 17 when viewed from the backside Ar3. Moreover, FIG. 14B is a cross-sectional view of an M-M line illustrated in FIG. 14A. Furthermore, FIG. 14C is a diagram illustrating the electrode 17 when viewed from the treatment portion side Ar4. FIGS. 15A to 15C correspond to FIGS. 5A to 5D, and are diagrams for explaining a configuration of the pad 14 according to the fourth modification example. That is, FIG. 15A is a diagram illustrating the pad 14 according to the fourth modification example when viewed from the backside Ar3. Moreover, FIG. 15B is a cross-sectional view of an N-N line illustrated in FIG. 15A. Furthermore, FIG. 15C is a diagram illustrating the pad 14 according to the fourth modification example when viewed from the treatment portion side Ar4. In the embodiment described earlier, the configuration of the gripper 12 can be changed as illustrated in FIGS. 12A to 15C.

As illustrated in FIGS. 12A to 12C, the gripper 12 according to the fourth modification example includes the jaw 13, the electrode 17, and the pad 14.

As illustrated in FIGS. 13A to 13C, in the jaw 13 according to the fourth modification example, the pair of jaw-side protruding portions 132 are not provided unlike in the jaw 13 according to the embodiment described earlier. Moreover, in the jaw 13 according to the fourth modification example, as illustrated in FIG. 13A and 13C, a pair of through holes 1315A are formed through both sides of the bottom wall portion 1315. More particularly, as illustrated in FIG. 13C, when viewed from the treatment portion side Ar4, the pair of through holes 1315A are formed at adjacent positions in the substantially central portion in the longitudinal direction along the central axis Ax2 on the mutually opposite faces of the sidewall portions 1313 and 1314. Thus, the pair of through holes 1315A are formed to be symmetrical with reference to the central axis Ax2.

The electrode 17 is configured using an electrically conductive material such as a metal, and functions as the electrode for supplying a high-frequency current to the treatment target. As illustrated in FIGS. 14A to 14C, the electrode 17 includes a pair of electrode-side protruding portions 171, a pair of first connecting portions 172, and a pair of second connecting portions 173.

As illustrated in FIGS. 14A to 14C, when viewed from the backside Ar3 or the treatment portion side Ar4, the pair of electrode-side protruding portions 171 are disposed to be symmetrical with reference to the central axis Ax2. More particularly, the electrode-side protruding portions 171 extend in a linear manner along the central axis Ax2. Moreover, as illustrated in FIG. 14B, the end face of each electrode-side protruding portion 171 on the treatment portion side Ar4 is made of an inclined face that becomes inclined toward the treatment portion side Ar4 as the distance from the other electrode-side protruding portion 171 goes on increasing.

As illustrated in FIGS. 14A and 14C, the pair of first connecting portions 172 connect the front end side Ar1 and the proximal end side Ar2 of the end portions of the pair of electrode-side protruding portions 171 on the backside Ar3.

The pair of second connecting portions 173 connect the electrode 17 to the jaw 13. As illustrated in FIGS. 14A and 14C, on the faces of the pair of electrode-side protruding portions 171 on the sides moving away from each other, the pair of second connecting portions 173 protrude from the substantially central portion of the end portion of the backside Ar3 toward the backside Ar3 in the longitudinal direction along the central axis Ax2. In the state of being inserted through the pair of through holes 1315A, the pair of second connecting portions 173 are fixed to the jaw 13. The fixing of the pair of second connecting portions 173 to the jaw 13 can be done using snap fitting, swaging, welding, or press fitting. In FIGS. 12A to 12C, the state in which the pair of second connecting portions 173 are fixed to the jaw 13 using swaging is illustrated.

In the pad 14 according to the fourth modification example, as illustrated in FIGS. 15A to 15C, the shape of the connecting portions 143 is different than the shape of the connecting portions 143 in the pad 14 according to the embodiment described earlier. The connecting portions 143 according to the fourth modification example connect the entire region in the longitudinal direction along the central axis Ax2 in the end portion of the abutting portion 141 on the backside Ar3 with the entire region in the longitudinal direction along the central axis Ax2 in the end portion of each pad-side protruding portion 142 on the backside Ar3. Thus, in the pad 14 according to the fourth modification example, the opening portions 144 according to the embodiment described earlier are not provided.

Moreover, in the pad 14 according to the fourth modification example, as illustrated in FIG. 15C, in the end portion of the abutting portion 141 on the treatment portion side Ar4, at the positions on the front end side Ar1 and the proximal end side Ar2, first notches 1411 are formed through which the pair of first connecting portions 172 are inserted.

Furthermore, in the pad 14 according to the fourth modification example, as illustrated in FIGS. 15A and 15C, in the substantially central portion of the pair of pad-side protruding portions 142 in the longitudinal direction along the central axis Ax2, second notches 1421 through which the pair of second connecting portions 173 are inserted are formed.

As illustrated in FIGS. 12A to 12C, the first connecting portions 172 are inserted in the first notches 1411, and the second connecting portions 173 are inserted in the second notches 1421; so that the pad 14 according to the fourth modification example is held in between the jaw 13 and the electrode 17. In that state, as illustrated in FIG. 12B, the end faces of the electrode-side protruding portions 171 on the treatment portion side Ar4 are positioned more toward the backside Ar3 with respect to the end faces of the sidewall portions 1311 to 1314 on the treatment portion side Ar4. Moreover, the end face of the abutting portion 141 on the treatment portion side Ar4 is positioned in between the inclined faces of the pair of electrode-side protruding portions 171 representing the end faces on the treatment portion side Ar4. Moreover, the end faces of the pad-side protruding portions 142 on the treatment portion side Ar4 are positioned on both sides of the pair of electrode-side protruding portions 171 in the width direction, and are positioned more toward the treatment portion side Ar4 with respect to the end faces of the sidewall portions 1311 to 1314 on the treatment portion side Ar4.

Even when the configuration according to the fourth modification example is adapted, it becomes possible to achieve identical effects to the effects achieved according to the embodiment described earlier.

The treatment tool and the pad according to the disclosure enable suppressing the sticking of the body tissue onto the gripper.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
an ultrasonic blade that includes a treatment face having a first face and having a second face adjacent to the first face, the ultrasonic blade being configured to supply ultrasonic vibration and high-frequency current from the treatment face to a body tissue;
a gripper that includes an electrode configured to open and close with respect to the ultrasonic blade to grip the body tissue between the gripper and the ultrasonic blade and supply the high-frequency current to the gripped body tissue;
an abutting portion that is made of an electrically insulating material and is disposed in the gripper, the abutting portion being configured to abut against the first face when the gripper closes with respect to the ultrasonic blade; and
a protruding portion that is made of an electrically insulating material, is spaced apart from the abutting portion in the gripper, and protrudes toward the second face,
a part of the electrode being exposed from a region between the abutting portion and the protruding portion and facing the ultrasonic blade, and
the abutting portion and the protruding portion forming a pad having an integrated structure.

2. The treatment tool according to claim 1, wherein the pad is fixed to the gripper with a pin.

3. The treatment tool according to claim 2, wherein the pin is press-fit or welded to the gripper.

4. The treatment tool according to claim 1, wherein a part of the electrode is exposed from front end side of the abutting portion and the protruding portion and faces the ultrasonic blade.

5. The treatment tool according to claim 1, wherein, in the gripper, a face not opposite to the ultrasonic blade is covered by an insulating portion made of an electrically insulating material.

6. The treatment tool according to claim 1, wherein, in the ultrasonic blade, a face not opposite to the gripper is coated by an electrically insulating material.

7. The treatment tool according to claim 1, wherein the protruding portion covers an end portion of the gripper in a width direction.

8. The treatment tool according to claim 1, wherein
the gripper includes
a jaw configured to open and close with respect to the ultrasonic blade, and
a holder configured to swing with respect to the jaw, and
the holder includes the electrode.

9. The treatment tool according to claim 1, wherein the pad is integrated with the gripper with welding or insert molding.

10. The treatment tool according to claim 1, wherein the gripper includes
a jaw configured to open and close with respect to the ultrasonic blade, and
the electrode that is attached to the jaw.

11. The treatment tool according to claim 10, wherein the pad is held in between the jaw and the electrode.

12. The treatment tool according to claim 11, wherein the electrode is fixed to the jaw with snap fitting, swaging, welding, or press fitting.

13. The treatment tool according to claim 1, wherein the protruding portion is disposed on each of both sides of the abutting portion in a width direction.

14. The treatment tool according to claim 1, wherein the pad includes guides on a front end side and a proximal end side, the guides being configured to abut against the ultrasonic blade to determine a relative position between the gripper and the ultrasonic blade in a width direction when the gripper closes with respect to the ultrasonic blade.

15. A pad made of an electrically insulating material, comprising:
an abutting portion configured to abut against an ultrasonic blade;
a protruding portion spaced apart from the abutting portion;
an opening portion that is formed in between the abutting portion and the protruding portion and from which a part of an electrode gets exposed; and
a connecting portion configured to connect the abutting portion and the protruding portion to each other.
